(19) United States
(12) Patent Application Publication
(10) Pub. No.: US 2009/0080967 A1
(43) Pub. Date: Mar. 26, 2009

(54) Joint and/or bearing arrangement

(76) Inventor: Matthias Gercke, Affinghausen (DE)

Correspondence Address: Raymond R. Ferrera, 1221 McKinney Street, Suite 4400, Houston, TX, 77010, US

(21) Appl. No.: 12/231,002

(22) Filed: Aug. 28, 2008

(30) Foreign Application Priority Data
Aug. 30, 2007 (DE) 10 2007 041 356.6

(51) Int. Cl. A61F 2/30; B60G 9/00

(52) U.S. Cl. 403/135; 280/124.1

(57) ABSTRACT

Joint or bearing arrangement (1) with a trunnion (2), the head (3) of which is moveable in relation to an accommodating joint socket (4), wherein the joint socket (4) is located in an accommodating cavity acting as a housing (5) when assembled and is moveable axially in relation to the latter, formed so that the joint socket (4) has a reinforcement (9) inhibiting its axial deformation.

### STATEMENT OF RELATED CASES

Pursuant to 35 U.S.C. 119(a), the instant application claims priority to prior German application number 10 2007 041 356.6, filed Aug. 30, 2007.

### DESCRIPTION OF THE INVENTION

The invention relates to a joint and/or bearing arrangement in accordance with the preamble to claim 1 and to a motor vehicle with one or more such joints and/or bearing arrangements, particularly in the chassis or steering components.

In joint arrangements which have a trunnion, the head of which is to be held moveably in a joint socket, it is favourable for said joint socket to be formed of a relatively soft material, in order to achieve sufficient damping and quiet operation of the joint or bearing arrangement. For example, a POM plastic or another thermoplastic is frequently used for a joint socket, entailing the difficulty that a joint socket in a plastic suitable for the specified comfort requirements frequently reaches its yield point at a high temperature, for example even 80° C., and/or under high radial stress, which may thus cause plastic and therefore irreversible deformation, such as thinning, of the joint socket, entailing play in the joint and a loss of strength in the joint socket. In particular, such radial stress may occur under powerful acceleration or braking in a motor vehicle. Apart from the physical stress, heat radiation from the brakes may intensify the problem.

In addition, it is necessary to render the joint or bearing socket axially moveable in relation to the axis of the joint compared to a surrounding accommodating cavity, particularly for moveable bearing arrangements, such as MacPherson suspension strut axles, which have a steering hub separate from the wheel hub. Such axial mobility must be ensured not only on installation, but also in operation of the moveable bearing.

The invention is based upon the problem of achieving a permanent improvement in the dimensional accuracy and strength of the joint socket even at a high temperature and/or under radial stress in joints with axially moveable joint sockets.

The invention solves this problem by means of a joint or bearing arrangement with the features of claim 1 and by means of a motor vehicle with the characteristics of claim 16. Reference is made to claims 2 to 15 and 17 in respect of advantageous embodiments and further developments of the invention.

The invention prevents axial creep of the joint or bearing socket material. Under ideal circumstances, the pressure exercised by the head of the trunnion on the peripheral walls of the joint or bearing socket under radial stress cannot lead to yield of the wall material in an axial direction, due to the axial reinforcement, and this effect is reduced in any case. The wall thickness therefore cannot decrease, preventing thinning of the material.

In particular, the reinforcement is fully embedded in the joint socket and enclosed by its material, so that its contact surfaces with the head of the trunnion on one hand and with the surrounding accommodating cavity on the other are unaffected by the reinforcement. A turning or tipping movement by the head of the trunnion in relation to the radial internal contact surface of the joint socket therefore remains possible without being affected, as does axial displaceability of the joint socket and its outer contact surface in relation to the surrounding accommodating cavity. Friction is unaffected by the reinforcement.

The reinforcement is advantageously extended along a significant section of the axial length of the joint socket to prevent creep effectively. The reinforcement is formed continuously with no weakness, particularly in the axially central area, in which the material of the joint socket, e.g. of a ball and socket joint, is at its thinnest.

The reinforcement may be formed, at least in part, by fibres, e.g. aramide or Teflon fibres, which are particularly good at preventing axial material creep of the joint socket material, if the fibres extend with an axial component and, for example, are distributed throughout the entire periphery of the joint socket.

At least some of the fibres may be favourably woven and/or knitted together, producing a stabilising mat and evenly distributing forces arising.

In addition, or alternatively, the reinforcement may comprise a dimensionally-stable insert, in one or more parts, separately or as a whole. This has, in particular, undercuts for as homogenous permeation as possible by the joint socket plastic. Cavities in the reinforcement may then be filled completely with plastic material.

It is then particularly advantageous for the insert to form a ring perpendicular to the axis, in the approximate shape of a crown and which has several through openings in the area facing away axially from the opening for the joint trunnion and several through openings in the area facing the opening for the joint trunnion axially.

In every case, the insert is formed from a material which is harder than the surrounding material of the joint socket, particularly a thermoplastic, e.g. steel or a strong plastic such as PEEK. A plastic insert can be co-extruded with the surrounding plastic of the joint socket.

Both in an inherently rigid component and in fibre reinforcement it may comprise barriers against axial deformation extending substantially perpendicular to the joint axis. Such barriers may point inwards to the joint axis in the form of necks and be penetrated by the latter, e.g. perpendicularly. The plane of the neck is then perpendicular to the joint axis, producing particularly effective inhibition of axial creep of the joint socket material.

The invention is particularly suitable for use in highly-stressed joints in a moveable bearing arrangement, e.g. in MacPherson suspension strut axles in vehicles with front-wheel drive, in which a static wheel hub is combined with a swivel wheel hub.

Further advantages and features of the invention emerge from the embodiments of the object of the invention shown in the drawings and described below.

### BRIEF DESCRIPTION OF THE DRAWINGS

In the drawings,

FIG. 1 is a diagrammatic partial general section through an inventive joint and/or bearing arrangement with an inherently dimensionally-stable insert in the joint socket;

FIG. 2 is an enlarged detail of a partially truncated joint socket accommodating the reinforcement and the moveable trunnion head shown in FIG. 1;

FIG. 3 is a separate drawing of a reinforcement in the form of a steel ring with apertures and a through slot;

FIG. 4 is a perspective view of an alternative reinforcement ring in the form of a crown;

FIG. 5 is a further alternative embodiment of a reinforcement ring with two through slots;

FIG. 6 is a horizontal projection of the reinforcement ring from FIG. 3;

FIG. 7 is a section along the line VII-VII in FIG. 6;

FIG. 8 is a similar view to FIG. 7, but after injection moulding of the reinforcement ring with the joint socket plastic;

FIG. 9 is a similar view to FIG. 8, but of an alternative embodiment with end areas of the reinforcement pointing outwards, showing only part of the ring as far as the axis of symmetry;

FIG. 10 is a similar view to FIG. 9, but of a further embodiment with a curved reinforcement adapted to the joint trunnion without separately curved end areas;

FIG. 11 is a similar view to FIG. 10, but of a further alternative embodiment with a reinforcement woven into a mesh fully permeating the joint socket;

FIG. 12 is a similar view to FIG. 11, but of a further alternative embodiment with individual fibres as reinforcement, arranged axially and parallel to each other;

FIG. 13 is a similar view to FIG. 12, but of a further alternative embodiment with a C-shaped reinforcement, as in FIG. 8, but formed by a fabric of a reinforcement phase.

### DETAILED DESCRIPTION OF THE INVENTION

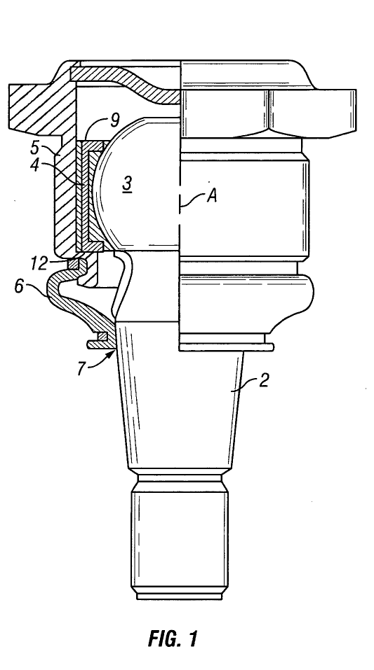

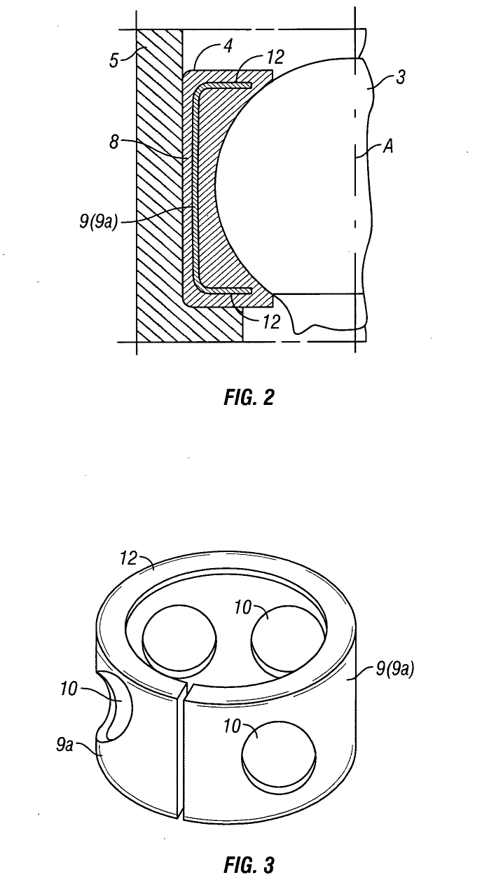

The joint arrangement 1 in accordance with FIG. 1 comprises an axially extended joint trunnion 2 with an extended head 3, for example in substantially spherical form. This may be retained so that it may turn and/or tip by a joint or bearing socket 4, usually permanently lubricated, accommodating the head. The joint socket 4 may be substantially closed apart from the trunnion entrance, or rather formed as a ring.

Furthermore, the bearing socket 4 is itself at least radially partly surrounded externally by an accommodation cavity acting as a housing 5. The housing 5 need not constitute a separate component, but may also be an integral component of, for example, a steering column encompassing the joint arrangement 1 in its assembled state. Such a steering column may be made, for example by extrusion coating or encapsulation, with a hot, liquid material, for example by zinc diecasting. wherein the steering rod is rigid and dimensionally-stable after subsequent cooling in a mould, and may form an integral housing 5 without further machining.

The joint and/or bearing arrangement 1 also comprises a sleeve gasket 6, frequently in the form of a sealing bellows. It seals the opening 7 for the trunnion 2 left in the housing 5 to the joint socket 4.

The joint and/or bearing arrangement 1 may during operation only be under radial stress or also use axial play Δs as room for movements in a moveable bearing arrangement. Such room for axial movements is also useful for assembly and dismantling.

The joint socket 4 may, for example, be formed mainly of a PA, POM, PBT, PEEK or cross-linked types of these materials (e.g. by electron beam cross-linking). They guarantee the necessary temperature resistance yet are sufficiently soft and elastically yielding, to provide high comfort and low noise in operation.

As forces acting radially outwards act on the peripheral walls 8 of the joint socket 4, due to the radial load of joint trunnion 3 in operation, so that they display the tendency to yield by creeping in an axial direction, particularly under heavy loads and/or at high temperatures, the joint socket 4 comprises reinforcement 9 inhibiting at least its axial deformation. This may take different forms.

The reinforcement 9 is integrally embedded in the plastic material of the joint socket 4. If, for example, the reinforcement 9 is formed of individual fibres 9g, the axial ends of the latter can terminate flush with the joint socket 4, so that they are visible from the outside.

The reinforcement 9 also ensures that is possible to absorb the axial and/or radial forces occurring in operation and to guarantee operation free of play and rattle. The desired turning and tipping seat of the head 3 in the joint socket 4 can thus simultaneously be guaranteed and the latter can remain displaceable axially in relation to the housing 5. The reinforcement 9 also makes it possible to introduce prestress into the joint socket 4 which remains at least almost constant throughout its useful life.

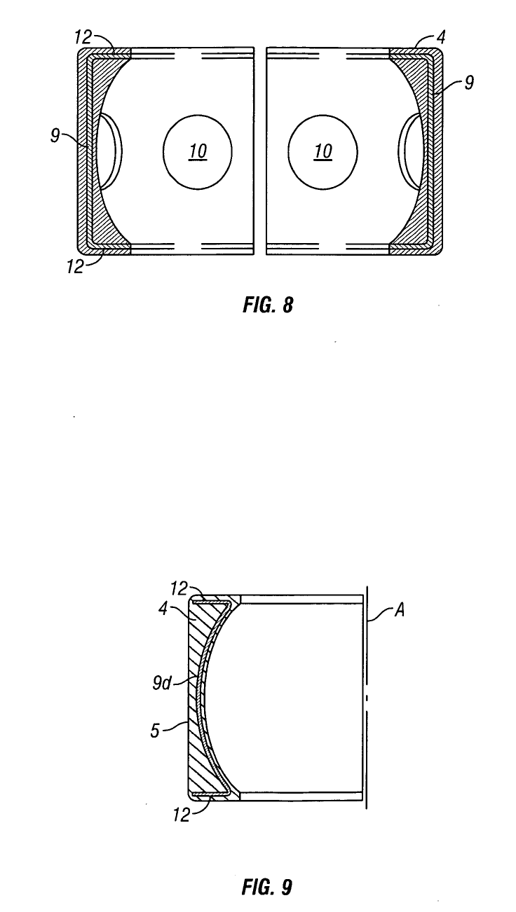

In the view shown in FIG. 1, the reinforcement takes the form of an inherently dimensionally-stable ring 9a with a C-shaped cross-section (FIG. 3, FIG. 7). This ring is enclosed radially inwards and outwards and axially by the plastic material of bearing socket 4, as may be seen, for example, from FIG. 8, thus forming an insert, in one piece in this case. An insert in more than one piece is also possible and is illustrated, for example, by the ring half-sockets 9b in FIG. 5.

In both cases, the insert 9a, 9b is formed of a harder material than the surrounding material of the joint socket 4, e.g. from steel.

The reinforcement 9 covers significantly more than 80% of the axial extension of the joint socket 4, in this case approximately 95%. It comprises undercuts or apertures 10 which are also permeated by the plastic material of the joint socket 4, in order to thus ensure intensive embedding and retention of the reinforcement 9.

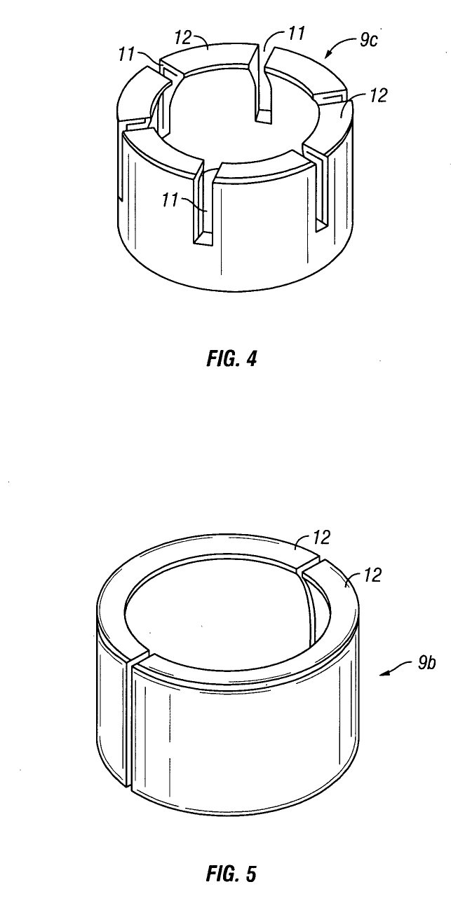

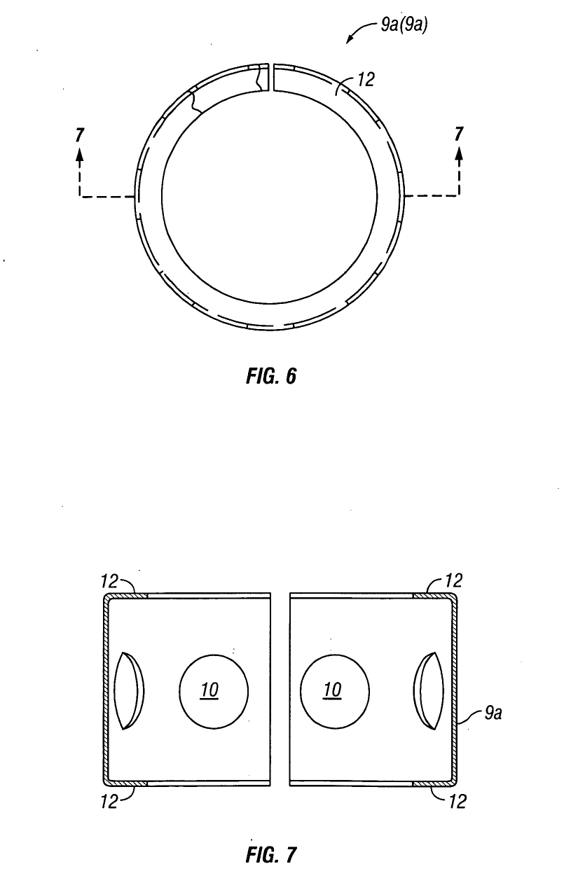

In an alternative embodiment in accordance with FIG. 4, the reinforcement 9 also forms a completely embedded ring 9c, which is itself perpendicular to the axis. This takes the form of a crown and has several recesses 11 in the form of slots in the area facing axially away from the opening 7 for the joint trunnion 2 and in the area axially facing the opening for the joint trunnion, to create individual spread wings.

Alternatively, the dimensionally-stable inserts 9a, 9b, 9c may also be formed of plastic and coextruded with the surrounding plastic of the joint socket to accelerate and simplify manufacture.

The ring-shaped reinforcement 9a, 9b, 9c is formed with a constant thickness without a weakened area, continuously in this case, at least outside its axial end areas.

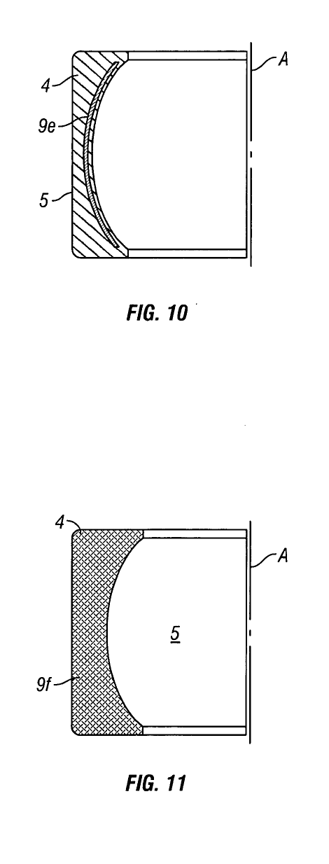

FIGS. 9 and 10 show further alternative dimensionally-stable rings 9d, 9e embedded in the joint socket 4 as the reinforcement 9. Both are adapted to the curvature of the contact surface of the joint socket 4 with the head 3. Like the rings 9a, 9b, 9c described above, the ring 9d contains integrally-formed barriers 12 against axial deformation extending substantially perpendicular to joint axis A. In rings 9a, 9b and 9c these barriers 12 are formed as necks of the reinforcement 9 and permeated by the latter, and pointing inwards to the joint axis. In contrast, in the case of curved ring 9d, these barrier-forming necks 12 point radially outwards from the axis A. In both cases, they cover approximately 10% of the diameter of the joint socket 4.

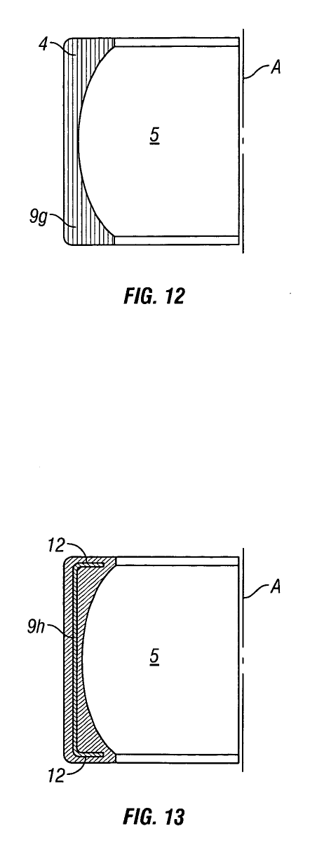

In addition or alternatively, the reinforcement 9 can also comprise fibres extending with an axial component, as indicated in FIGS. 11 to 13.

In the embodiment in FIG. 9, the fibres of the reinforcement 9 are woven and/or knitted together to form a mat 9f and permeate the entire joint socket 4 throughout their axial extension.

In the illustration shown in FIG. 10, the fibres 9g extend parallel to the axis A and to each other and spaced apart from each other. In contrast, in FIG. 13, a C-shaped ring 9h is formed similarly to that in FIG. 1 but as a weave or knit, e.g. of glass, carbon or nylon fibres, not as a steel ring.

A combination of fibre and solid reinforcement is also possible.

Such joint and/or bearing arrangements 1 may be used advantageously in their inventive embodiment, particularly in chassis and/or steering components of motor vehicles, also, for example, to compensate structural tolerance between the steering column and the wheel hub on installation in the vehicle. In particular, such joint and/or bearing arrangements 1 may form a moveable bearing within a MacPherson suspension strut axle of a vehicle with at least front-wheel drive, in which a sprung wheel hub and a second hub providing steerability are formed separately and the second hub is held on the wheel trunk by a moveable bearing with axial mobility.

### LIST OF REFERENCE NUMBERS

1. Joint or bearing arrangement
2. Trunnion
3. Head
4. Joint socket
5. Housing
6. Sleeve gasket
7. Opening
8. Peripheral walls
9. Reinforcement
10. Undercuts or apertures
11. Recesses
12. Barrier area
A Axis

1. A joint and/or bearing arrangement (1) with a trunnion (2), the head (3) of which is moveable in relation to an accommodating joint socket (4), wherein the joint socket (4) is located in an accommodating cavity acting as a housing (5) when assembled and is axially moveable in relation to the latter, wherein said joint socket (4) further comprises a reinforcement (9) inhibiting its axial deformation.

2. The joint and/or bearing arrangement (1) in accordance with claim 1, wherein said reinforcement (9) is embedded in the joint socket (4).

3. The joint and/or bearing arrangement (1) in accordance with claim 1, wherein said reinforcement (9) covers at least 80% of the axial extension of the joint socket (4).

4. The joint and/or bearing arrangement (1) in accordance with claim 1, wherein said reinforcement (9) is formed with a constant thickness, at least outside its axial end areas.

5. The joint and/or bearing arrangement (1) in accordance with claim 1, wherein said reinforcement (9) comprises fibres (9f; 9g; 9h) extending with an axial component.

6. The joint and/or bearing arrangement (1) in accordance with claim 5, wherein said fibres (9f, 9h) are woven and/or knitted together to form a meshwork.

7. The joint and/or bearing arrangement (1) in accordance with claim 1, wherein said reinforcement (9) comprises a dimensionally-stable insert (9a; 9b; 9c; 9d; 9e) in one or more parts.

8. The joint and/or bearing arrangement (1) in accordance with claim 7, wherein said insert (9c) forms a ring perpendicular to the axis (A) which has several recesses (11) in the area facing axially away from the opening (7) for the joint trunnion (2) and in the area axially facing the opening (7) for the joint trunnion.

9. The joint and/or bearing arrangement (1) in accordance with claim 7, wherein said insert (9a; 9b; 9c; 9d; 9e) is formed of a harder material than the surrounding material of the joint socket (4).

10. The joint and/or bearing arrangement (1) in accordance with claim 7 wherein said insert (9a; 9b; 9c; 9d; 9e) is formed of steel.

11. The joint and/or bearing arrangement (1) in accordance with claim 7, wherein said insert (9a; 9b; 9c; 9d; 9e) is formed of plastic and co-extruded with the surrounding plastic of the joint socket (4).

12. The joint and/or bearing arrangement (1) in accordance with claim 7, wherein said insert (9a; 9b; 9c; 9d; 9e) comprises undercuts (10) in which the plastic of the joint socket (4) engages.

13. The joint and/or bearing arrangement (1) in accordance with claim 1, wherein said reinforcement (9) comprises barriers (12) against axial deformation extending substantially perpendicular to the joint axis (A).

14. The joint and/or bearing arrangement (1) in accordance with claim 13, wherein said barriers (12) are embodied as flange regions (12) of the reinforcement (9) which point inwards to the joint axis (A) by which they are permeated.

15. The joint and/or bearing arrangement (1) in accordance with claim 14, wherein said barriers (12) cover at least 10% of the diameter of the joint socket (4).

16. A motor vehicle with at least one joint and/or bearing arrangement (1) in accordance with claim 1, particularly in chassis and/or steering components.

17. The motor vehicle in accordance with claim 16, wherein said joint and/or bearing arrangement (1) forms a moveable bearing in a suspension strut axle of a vehicle with at least front-wheel drive, in which a sprung wheel hub and a second hub effecting steerability are formed separately and the second hub is held on the wheel hub by a moveable bearing.
